SEQUENCE LISTING (6 sequences)

SEQ ID NO: 1 - DNA, 20 bp, Artificial
  Oligonucleotide primer.
tctccctgatcggtgacagt

SEQ ID NO: 2 - DNA, 23 bp, Artificial
  Oligonucleotide primer.
tcgaaaaactgcactagagacaa

SEQ ID NO: 3 - DNA, 21 bp, Artificial
  Oligonucleotide primer.
gggaatcttctggaagacctg

SEQ ID NO: 4 - DNA, 20 bp, Artificial
  Oligonucleotide primer.
tacacacaacagcaccagca

SEQ ID NO: 5 - DNA, 20 bp, Artificial
  Oligonucleotide primer.
gtgtttgccaggcttaagga

SEQ ID NO: 6 - DNA, 20 bp, Artificial
  Oligonucleotide primer.
tcggtgtttccttcttccac

(19) United States
(12) Patent Application Publication
(10) Pub. No.: US 2010/0221722 A1
(43) Pub. Date: Sep. 2, 2010

(54) METHODS FOR EVALUATING BREAST CANCER PROGNOSIS

(75) Inventors: Charles M. Perou, Carrboro, NC (US); Zhiyuan Hu, Apex, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: UNIVERSITY OF NORTH CAROLINA AT CHAPEL HILL, CHAPEL HILL, NC (US)

(21) Appl. No.: 12/664,869

(22) PCT Filed: Jun. 13, 2008
(86) PCT No.: PCT/US08/66815

§ 371 Date: May 18, 2010

Related U.S. Application Data
(60) Provisional application No. 60/944,414, filed on Jun. 15, 2007.

(51) Int. Cl. C12Q 1/68; C12Q 1/02

(52) U.S. Cl. 435/6; 435/29

(57) ABSTRACT

Methods for diagnosing and for evaluating the prognosis of a cancer patient, particularly a breast cancer patient, are provided. The methods include determining expression levels of at least five biomarkers in a body sample including a cancer cell from the patient, where expression levels of the biomarkers are indicative of cancer prognosis. Overexpression of the biomarkers of the invention is indicative of a poor prognosis. In some embodiments, the body sample is a breast tissue sample, particularly a primary breast tumor sample. The methods of the invention can be used in combination with assessment of conventional clinical factors and permit a more accurate evaluation of breast cancer prognosis.

### FEDERALLY SPONSORED RESEARCH OR DEVELOPMENT

This invention was made with government support under grant numbers P50-CA58223-09A1 and RO1-CA-101227-01, awarded by the National Institutes of Health/National Cancer Institute. The United States government has certain rights in the invention.

### FIELD OF THE INVENTION

The present invention relates to methods for diagnosing and for evaluating the prognosis of a patient afflicted with breast cancer.

### BACKGROUND OF THE INVENTION

Breast cancer is the second most common cancer among women in the United States, second only to skin cancer. A woman in the U.S. has a one in eight chance of developing breast cancer during her lifetime, and the American Cancer Society estimates that more than 300,000 new cases of breast cancer will be reported in the U.S. in 2007. Breast cancer is the second leading cause of cancer deaths in women, with more than 40,000 deaths annually. Improved detection methods, mass screening, and advances in treatment over the last decade have significantly improved the outlook for woman diagnosed with breast cancer. Today, approximately 80% of breast cancer cases are diagnosed in the early stages of the disease when survival rates are at their highest. As a result, about 85% percent of breast cancer patients are alive at least five years after diagnosis. Despite these advances, approximately 20% of women diagnosed with early-stage breast cancer have a poor ten-year outcome and will suffer disease recurrence, metastasis or death within this time period.

Significant research has focused on identifying methods and factors for assessing breast cancer prognosis and predicting therapeutic response. (See generally, Ross and Hortobagyi, eds. (2005) Molecular Oncology of Breast Cancer (Jones and Bartlett Publishers, Boston, Mass.) and the references cited therein). Prognostic indicators include conventional factors, such as tumor size, nodal status and histological grade, as well as molecular markers that provide some information regarding prognosis and likely response to particular treatments. For example, determination of estrogen (ER) and progesterone (PR) steroid hormone receptor status has become a routine procedure in assessment of breast cancer patients. See, for example, Fitzgibbons et al., Arch. Pathol. Lab. Med. 124:966-78, 2000. Tumors that are hormone receptor positive are more likely to respond to hormone therapy and also typically grow less aggressively, thereby resulting in a better prognosis for patients with ER+/PR+ tumors. Overexpression of human epidermal growth factor receptor 2 (HER-2/neu), a transmembrane tyrosine kinase receptor protein, has been correlated with poor breast cancer prognosis (see, e.g., Ross et al., The Oncologist 8:307-25, 2003), and Her-2 expression levels in breast tumors are used to predict response to the anti-Her-2 monoclonal antibody therapeutic trastuzumab (Herceptin®, Genentech, South San Francisco, Calif.).

Despite recent advances, the challenge of cancer treatment remains to target specific treatment regimens to pathogenically distinct tumor types, and ultimately personalize tumor treatment in order to maximize outcome. In particular, once a patient is diagnosed with cancer, such as breast cancer, there is a need for methods that allow the physician to predict the expected course of disease, including the likelihood of cancer recurrence, long-term survival of the patient and the like, and select the most appropriate treatment options accordingly. Such methods should specifically distinguish breast cancer patients with a poor prognosis from those with a good prognosis and permit the identification of high-risk, early-stage breast cancer patients who are likely to need aggressive therapy.

### SUMMARY OF THE INVENTION

Methods for diagnosing and for evaluating the prognosis of a cancer patient, particularly a breast cancer patient, are provided. The methods include determining expression levels of at least five biomarkers selected from a group of biomarkers that includes RRAGD, FABP5, UCHL1, GAL, PLOD, DDIT4, VEGF, ADM, ANGPTL4, NDRG1, NP, SLC16A3, and C14ORF58 in a sample including a cancer cell or a tumor cell from the patient, where expression levels of the biomarkers are indicative of cancer prognosis. Overexpression of the biomarkers of the invention is indicative of a poor prognosis, that is, a high likelihood of cancer recurrence, metastasis or death from the underlying cancer.

In one embodiment, all thirteen of the biomarkers can be used for diagnosing and for evaluating the prognosis of a breast cancer patient. Furthermore, as new biomarkers are discovered or determined to be useful in the methods of the invention, they can be added for use in the analyses described herein.

Thus, the present methods permit the differentiation of breast cancer patients with a good prognosis from those patients with a poor prognosis. The methods disclosed herein can be used in combination with assessment of conventional clinical factors, such as tumor size, tumor grade, lymph node status, family history, and analysis of the expression level of additional biomarkers, such as Her-2 and estrogen and progesterone hormone receptors. In this manner, the methods of the invention permit a more accurate evaluation of breast cancer prognosis. The methods can also be used to plan a treatment regimen for patients, as those patients with a poor prognosis can receive more aggressive treatment options.

Methods of the invention include means for monitoring gene or protein expression, including gene arrays, polymerase chain reaction (PCR), antibody-based detection, and proteomics. Biomarker expression can be assessed at the protein or nucleic acid level. Kits comprising reagents for practicing the methods of the invention are provided.

### DETAILED DESCRIPTION OF THE INVENTION

### Overview

The present invention provides methods for diagnosing and for evaluating the prognosis of a cancer patient, particularly a breast cancer patient. Early diagnosis of breast cancer is essential to assure the best treatment results. The methods include detecting expression of and/or determining the expression levels of the RNA transcripts, or their expression products, of biomarkers in a patient sample (e.g., a tissue or body fluid sample) having a cancer cell. The biomarkers of the invention include RRAGD, FABP5, UCHL1, GAL, PLOD, DDIT4, VEGF, ADM, ANGPTL4, NDRG1, NP, SLC16A3, and C14ORF58.

In one embodiment, the method includes determining the expression levels of the RNA transcripts or their expression products of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample having a cancer cell from the patient. Biomarker expression in some instances may be normalized against the expression levels of all RNA transcripts or their expression products in the sample, or against a reference set of RNA transcripts or their expression products in the sample. The level of expression of the biomarkers is indicative of prognosis. In a specific, non-limiting example, overexpression of at least five biomarkers is indicative of poor breast cancer prognosis.

In another embodiment, the method includes detecting expression of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample from the patient, where overexpression of the biomarkers is indicative of a poor prognosis.

In a further embodiment, the method includes determining the expression levels of the RNA transcripts or their expression products of a set of biomarkers comprising RRAGD, FABP5, UCHL1, GAL, PLOD, DDIT4, VEGF, ADM, ANGPTL4, NDRG1, NP, SLC16A3, and C14ORF58 in a sample having a cancer cell from the patient, normalized against the expression levels of all RNA transcripts or their expression products in the sample, or of a reference set of RNA transcripts or their expression products in the sample, where expression of said set of biomarkers is indicative of prognosis. In a specific, non-limiting example, overexpression of at least five biomarkers is indicative of poor breast cancer prognosis.

The methods of the invention can also be used to assist in selecting appropriate courses of treatment and to identify patients that would benefit from more aggressive therapy. Thus, overexpression of a particular combination of at least five biomarkers of interest permits the differentiation of breast cancer patients that are likely to experience disease recurrence (i.e., poor prognosis) from those who are more likely to remain cancer-free (i.e., good prognosis).

By “breast cancer” is intended, for example, those conditions classified by biopsy as malignant pathology. The clinical delineation of breast cancer diagnoses is well-known in the medical arts. One of skill in the art will appreciate that breast cancer refers to any malignancy of the breast tissue, including, for example, carcinomas and sarcomas. In particular embodiments, the breast cancer is ductal carcinoma in situ (DCIS), lobular carcinoma in situ (LCIS), or mucinous carcinoma. Breast cancer also refers to infiltrating ductal (IDC) or infiltrating lobular carcinoma (ILC). In most embodiments of the invention, the subject of interest is a human patient suspected of or actually diagnosed with breast cancer.

The American Joint Committee on Cancer (AJCC) has developed a standardized system for breast cancer staging using a “TNM” classification scheme. Patients are assessed for primary tumor size (T), regional lymph node status (N), and the presence/absence of distant metastasis (M) and then classified into stages 0-IV based on this combination of factors. In this system, primary tumor size is categorized on a scale of 0-4 (T0: no evidence of primary tumor; T1:≦2 cm; T2:>2 cm-≦5 cm; T3:>5 cm; T4: tumor of any size with direct spread to chest wall or skin). Lymph node status is classified as N0-N3 (N0: regional lymph nodes are free of metastasis; N1: metastasis to movable, same-side axillary lymph node(s); N2: metastasis to same-side lymph node(s) fixed to one another or to other structures; N3: metastasis to same-side lymph nodes beneath the breastbone). Metastasis is categorized by the absence (M0) or presence of distant metastases (M1). Methods of identifying breast cancer patients and staging the disease are well known and may include manual examination, biopsy, review of patient's and/or family history, and imaging techniques, such as mammography, magnetic resonance imaging (MRI), and positron emission tomography (PET).

The term “prognosis” is recognized in the art and encompasses predictions about the likely course of disease or disease progression, particularly with respect to likelihood of disease remission, disease relapse, tumor recurrence, metastasis, and death. “Good prognosis” refers to the likelihood that a patient afflicted with cancer, particularly breast cancer, will remain disease-free (i.e., cancer-free). “Poor prognosis” is intended to mean the likelihood of a relapse or recurrence of the underlying cancer or tumor, metastasis, or death. Cancer patients classified as having a “good outcome” remain free of the underlying cancer or tumor. In contrast, “bad outcome” cancer patients experience disease relapse, tumor recurrence, metastasis, or death. In particular embodiments, the time frame for assessing prognosis and outcome is, for example, less than one year, one, two, three, four, five, six, seven, eight, nine, ten, fifteen, twenty, or more years. As used herein, the relevant time for assessing prognosis or disease-free survival time begins with the surgical removal of the tumor or suppression, mitigation, or inhibition of tumor growth. Thus, for example, in particular embodiments, a “good prognosis” refers to the likelihood that a breast cancer patient will remain free of the underlying cancer or tumor for a period of at least five, such as for a period of at least ten years. In further aspects of the invention, a “poor prognosis” refers to the likelihood that a breast cancer patient will experience disease relapse, tumor recurrence, metastasis, or death within less than ten years, such as less than five years. Time frames for assessing prognosis and outcome provided herein are illustrative and are not intended to be limiting.

In some embodiments described herein, prognostic performance of the biomarkers and/or other clinical parameters was assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., overexpression of particular biomarkers, as described herein). Cox model data are commonly presented as Kaplan-Meier curves or plots. The “hazard ratio” is the risk of death at any given time point for patients displaying particular prognostic variables. See generally Spruance et al., Antimicrob. Agents & Chemo. 48:2787-92, 2004. In particular embodiments, the biomarkers of interest are statistically significant for assessment of the likelihood of breast cancer recurrence or death due to the underlying breast cancer. Methods for assessing statistical significance are well known in the art and include, for example, using a log-rank test, Cox analysis and Kaplan-Meier curves. In some aspects of the invention, a p-value of less than 0.05 constitutes statistical significance.

As described herein, a number of clinical and prognostic breast cancer factors are known in the art and are used to predict treatment outcome and the likelihood of disease recurrence. Such factors include, for example, lymph node involvement, tumor size, histologic grade, family history, estrogen and progesterone hormone receptor status, Her-2 levels, and tumor ploidy. As used herein, estrogen and progesterone hormone receptor status refers to whether these receptors are expressed in the breast tumor of a particular breast cancer patient. Thus, an “estrogen receptor-positive patient” displays ER expression in a breast tumor, whereas an “estrogen receptor-negative patient” does not. Using the methods of the present invention, the prognosis of a breast cancer patient can be determined independent of or in combination with assessment of these or other clinical and prognostic factors. In some embodiments, combining the methods disclosed herein with evaluation of other prognostic factors may permit a more accurate determination of breast cancer prognosis. The methods of the invention may be coupled with analysis of, for example, Her-2 expression levels. Other factors, such as patient clinical history, family history and menopausal status, may also be considered when evaluating breast cancer prognosis via the methods of the invention. In some embodiments, patient data obtained via the methods disclosed herein may be coupled with analysis of clinical information and existing tests for breast cancer prognosis to develop a reference laboratory prognostic algorithm. Such algorithms find used in stratifying breast cancer patients, particularly early-stage breast cancer patients, into good and poor prognosis populations. Patients assessed as having a poor prognosis may be upstaged for more aggressive breast cancer treatment.

Breast cancer is managed by several alternative strategies that may include, for example, surgery, radiation therapy, hormone therapy, chemotherapy, or some combination thereof. As is known in the art, treatment decisions for individual breast cancer patients can be based on endocrine responsiveness of the tumor, menopausal status of the patient, the location and number of patient lymph nodes involved, estrogen and progesterone receptor status of the tumor, size of the primary tumor, patient age, and stage of the disease at diagnosis. Analysis of a variety of clinical factors and clinical trials has led to the development of recommendations and treatment guidelines for early-stage breast cancer by the International Consensus Panel of the St. Gallen Conference (2005). See, Goldhirsch et al., Annals Oncol. 16:1569-83, 2005. The guidelines recommend that patients be offered chemotherapy for endocrine non-responsive disease; endocrine therapy as the primary therapy for endocrine responsive disease, adding chemotherapy for some intermediate- and all high-risk groups in this category; and both chemotherapy and endocrine therapy for all patients in the uncertain endocrine response category except those in the low-risk group. Stratification of patients into poor prognosis or good prognosis risk groups at the time of diagnosis using the methods disclosed herein provides an additional or alternative treatment decision-making factor. The methods of the invention permit the differentiation of breast cancer patients with a good prognosis from those more likely to suffer a recurrence (i.e., patients who might need or benefit from additional aggressive treatment at the time of diagnosis).

The methods of the invention find particular use in choosing appropriate treatment for early-stage breast cancer patients. The majority of breast cancer patients diagnosed at an early-stage of the disease enjoy long-term survival following surgery and/or radiation therapy without further adjuvant therapy. However, a significant percentage (approximately 20%) of these patients will suffer disease recurrence or death, leading to clinical recommendations that some or all early-stage breast cancer patients should receive adjuvant therapy (e.g., chemotherapy). The methods of the present invention find use in identifying this high-risk, poor prognosis population of early-stage breast cancer patients and thereby determining which patients would benefit from continued and/or more aggressive therapy and close monitoring following treatment. For example, early-stage breast cancer patients assessed as having a poor prognosis by the methods disclosed herein may be selected for more aggressive adjuvant therapy, such as chemotherapy, following surgery and/or radiation treatment. In particular embodiments, the methods of the present invention may be used in conjunction with the treatment guidelines established by the St. Gallen Conference to permit physicians to make more informed breast cancer treatment decisions.

The present methods for evaluating breast cancer prognosis can also be combined with other prognostic methods (e.g., assessment of conventional clinical factors, such as tumor size, tumor grade, lymph node status, and family history) additional molecular markers known in the art (e.g., estrogen and progesterone hormone receptors, Her-2 and p53) and additional microarrays (e.g., Agilent (van't Veer et al., N. Engl. J. Med. 347:1999-2009, 2002) and Affymetrix (Pawitan et al., Cancer Res. 7: 953-64, 2005)) for purposes of selecting an appropriate breast cancer treatment. By “microarray” is intended an ordered arrangement of hybridizable array elements, such as, for example, polynucleotide probes, on a substrate.

The methods disclosed herein also find use in predicting the response of a breast cancer patient to a selected treatment. By “predicting the response of a breast cancer patient to a selected treatment” is intended assessing the likelihood that a patient will experience a positive or negative outcome with a particular treatment. As used herein, “indicative of a positive treatment outcome” refers to an increased likelihood that the patient will experience beneficial results from the selected treatment (e.g., complete or partial remission, reduced tumor size, etc.). By “indicative of a negative treatment outcome” is intended an increased likelihood that the patient will not benefit from the selected treatment with respect to the progression of the underlying breast cancer. In some aspects of the invention, the selected treatment is chemotherapy. In other aspects of the invention, the selected treatment is anti-VEGF therapy, such as, for example, monoclonal antibody therapy (e.g., bevacizumab). In still other aspects of the invention, the selected treatment is anti-HIF1α therapy, such as, for example, treatment with small molecule inhibitors of HIF1α activity (see, e.g., Powis and Kirkpatrick, Mol. Cancer. Therap. 3:647-54, 2004).

In certain embodiments, methods for predicting the likelihood of survival of a breast cancer patient are provided. In particular, the methods may be used predict the likelihood of long-term, disease-free survival. By “predicting the likelihood of survival of a breast cancer patient” is intended assessing the risk that a patient will die as a result of the underlying breast cancer. “Long-term, disease-free survival” is intended to mean that the patient does not die from or suffer a recurrence of the underlying breast cancer within a period of at least five years, such as at least ten or more years, following initial diagnosis or treatment. Such methods for predicting the likelihood of survival of a breast cancer patient include detecting expression of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample from the patient, where overexpression of the biomarkers is indicative of a poor likelihood of survival. Likelihood of survival can be assessed in comparison to, for example, breast cancer survival statistics available in the art.

### Biomarkers

The biomarkers of the invention include genes and proteins. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Fragments and variants of biomarker genes and proteins are also encompassed by the present invention. By “fragment” is intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs.

A “biomarker” is a gene or protein whose level of expression in a tissue or cell is altered compared to that of a normal or healthy cell or tissue. The biomarkers of the present invention are genes and proteins whose overexpression correlates with cancer, particularly breast cancer, prognosis. As used herein, “overexpression” means expression greater than the expression detected in normal, non-cancerous tissue. For example, an RNA transcript or its expression product that is overexpressed in a cancer cell or tissue may be expressed at a level that is 1.5 times higher than in a in normal, non-cancerous cell or tissue, such as 2 times higher, 3 times higher, 5 times higher, or or more times higher.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their products). Normalization is performed to correct for or normalize away both differences in the amount of RNA assayed and variability in the quality of the RNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed biomarkers or a large subset thereof (global normalization approach).

In particular embodiments, selective overexpression of a biomarker or combination of biomarkers of interest in a patient sample is indicative of a poor cancer prognosis. By “indicative of a poor prognosis” is intended that overexpression of the particular biomarker or combination of biomarkers is associated with an increased likelihood of relapse or recurrence of the underlying cancer or tumor, metastasis or death. For example, “indicative of a poor prognosis” may refer to an increased likelihood of relapse or recurrence of the underlying cancer or tumor, metastasis, or death within ten years, such as five years. In other aspects of the invention, the absence of overexpression of a biomarker or combination of biomarkers of interest is indicative of a good prognosis. As used herein, “indicative of a good prognosis” refers to an increased likelihood that the patient will remain cancer-free. In some embodiments, “indicative of a good prognosis” refers to an increased likelihood that the patient will remain cancer-free for ten years, such as five years.

The biomarkers of the present invention are selected from the group consisting of RRAGD (Ras-related GTP binding D; GenBank Accession No. BC003088), FABP5 (fatty acid binding protein 5; GenBank Accession No. M94856), UCHL1 (ubiquitin carboxyl-terminal esterase L1; GenBank Accession No. NM_{—}004181), GAL (galanin; GenBank Accession No. BC030241), PLOD (procollagen-lysine, 2-oxoglutarate 5-dioxygenase lysine hydroxylase; GenBank Accession No. M98252), DDIT4 (DNA-damage-inducible transcript 4; GenBank Accession No. NM_{—}019058), VEGF (vascular endothelial growth factor; GenBank Accession No. M32977), ADM (adrenomedullin; GenBank Accession No. NM_{—}001124), ANGPTL4 (angiopoietin-like 4; GenBank Accession No. AF202636), NDRG1 (N-myc downstream regulated gene 1; GenBank Accession No. NM_{—}006096), NP (nucleoside phosphorylase; GenBank Accession No. NM 000270), SLC16A3 (solute carrier family 16 monocarboxylic acid transporters, member 3; GenBank Accession No. NM_{—}004207), and C14ORF58 (chromosome 14 open reading frame 58; GenBank Accession No. AK000378). Although the methods of the invention require the detection of at least five biomarkers in a patient sample for evaluating breast cancer prognosis, 6, 7, 8, 9, 10, 11, 12, 13, or more biomarkers may be used to practice the present invention.

### Sample Source

In particular embodiments, the methods for evaluating breast cancer prognosis include collecting a patient body sample having a cancer cell or tissue, such as a breast tissue sample or a primary breast tumor tissue sample. By “body sample” is intended any sampling of cells, tissues, or bodily fluids in which expression of a biomarker can be detected. Examples of such body samples include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the body sample includes breast cells, particularly breast tissue from a biopsy, such as a breast tumor tissue sample. Body samples may be obtained from a patient by a variety of techniques including, for example, by scraping or swabbing an area, by using a needle to aspirate cells or bodily fluids, or by removing a tissue sample (i.e., biopsy). Methods for collecting various body samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Body samples, particularly breast tissue samples, may be transferred to a glass slide for viewing under magnification. In one embodiment, the body sample is a formalin-fixed, paraffin-embedded breast tissue sample, particularly a primary breast tumor sample.

Any methods available in the art for detecting expression of biomarkers are encompassed herein. The expression of a biomarker of the invention can be detected on a nucleic acid level (e.g., as an RNA transcript) or a protein level. By “detecting expression” is intended determining the quantity or presence of an RNA transcript or its expression product of a biomarker gene. Thus, “detecting expression” encompasses instances where a biomarker is determined not to be expressed, not to be detectably expressed, expressed at a low level, expressed at a normal level, or overexpressed. In order to determine overexpression, the body sample to be examined can be compared with a corresponding body sample that originates from a healthy person. That is, the “normal” level of expression is the level of expression of the biomarker in, for example, a breast tissue sample from a human subject or patient not afflicted with breast cancer. Such a sample can be present in standardized form. In some embodiments, determination of biomarker overexpression requires no comparison between the body sample and a corresponding body sample that originates from a healthy person. For example, detection of overexpression of a biomarker indicative of a poor prognosis in a breast tumor sample may preclude the need for comparison to a corresponding breast tissue sample that originates from a healthy person. Moreover, in some aspects of the invention, no expression, underexpression, or normal expression (i.e., the absence of overexpression) of a biomarker or combination of biomarkers of interest provides useful information regarding the prognosis of a breast cancer patient.

Methods for detecting expression of the biomarkers of the invention, that is, gene expression profiling, include methods based on hybridization analysis of polynucleotides, methods based on sequencing of polynucleotides, immunohistochemistry methods, and proteomics-based methods. The most commonly used methods known in the art for the quantification of mRNA expression in a sample include northern blotting and in situ hybridization (Parker and Barnes, Methods Mol. Biol. 106:247-83, 1999), RNAse protection assays (Hod, Biotechniques 13:852-54, 1992), PCR-based methods, such as reverse transcription PCR(RT-PCR) (Weis et al., TIG 8:263-64, 1992), and array-based methods (Schena et al., Science 270:467-70, 1995). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE) and gene expression analysis by massively parallel signature sequencing.

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

### Hybridization Analysis Of Polynucleotides

In some embodiments, the expression of a biomarker of interest is detected at the nucleic acid level. Nucleic acid-based techniques for assessing expression are well known in the art and include, for example, determining the level of biomarker RNA transcripts (i.e., mRNA) in a body sample. Many expression detection methods use isolated RNA. The starting material is typically total RNA isolated from a body sample, such as a tumor or tumor cell line, and corresponding normal tissue or cell line, respectively. Thus RNA can be isolated from a variety of primary tumors, including breast, lung, colon, prostate, brain, liver, kidney, pancreas, spleen, thymus, testis, ovary, uterus, and the like, or tumor cell lines. If the source of mRNA is a primary tumor, mRNA can be extracted, for example, from frozen or archived paraffin-embedded and fixed (e.g., formalin-fixed) tissue samples.

General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155).

Isolated mRNA can be used in hybridization or amplification assays that include, but are not limited to, Southern or Northern analyses, PCR analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250, or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent gene chip array. A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, for example, by RT-PCR (U.S. Pat. No. 4,683,202), ligase chain reaction (Barany, Proc. Natl. Acad. Sci. USA 88:189-93, 1991), self sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). For PCR analysis, well known methods are available in the art for the determination of primer sequences for use in the analysis.

Biomarker expression levels of RNA may be monitored using a membrane blot (such as used in hybridization analysis such as Northern, Southern, dot, and the like), or microwells, sample tubes, gels, beads, or fibers (or any solid support comprising bound nucleic acids). See, for example, U.S. Pat. Nos. 5,770,722, 5,874,219, 5,744,305, 5,677,195 and 5,445,934. The detection of biomarker expression may also comprise using nucleic acid probes in solution.

In one embodiment of the invention, microarrays are used to detect biomarker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, PCR amplified inserts of cDNA clones are applied to a substrate in a dense array. For example, at least 10,000 nucleotide sequences are applied to the substrate. The microarrayed genes, immobilized on the microchip at 10,000 elements each, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink-jet microarray technology. The development of microarray methods for large-scale analysis of gene expression makes it possible to search systematically for molecular markers of cancer classification and outcome prediction in a variety of tumor types.

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, Velculescu et al. (Science 270:484-87, 1995; Cell 88:243-51, 1997).

An additional method of biomarker expression analysis at the nucleic acid level is gene expression analysis by massively parallel signature sequencing (MPSS), as described by Brenner et al. (Nat. Biotech. 18:630-34, 2000). This is a sequencing approach that combines non-gel-based signature sequencing with in vitro cloning of millions of templates on separate 5 μM diameter microbeads. First, a microbead library of DNA templates is constructed by in vitro cloning. This is followed by the assembly of a planar array of the template-containing microbeads in a flow cell at a high density (typically greater than 3.0×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

### Immunohistochemistry

Immunohistochemistry methods are also suitable for detecting the expression levels of the biomarkers of the present invention. In one embodiment, a patient breast tissue sample is collected by, for example, biopsy techniques known in the art. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In some instances, samples may need to be modified in order to make the biomarker antigens accessible to antibody binding. For example, formalin fixation of tissue samples results in extensive cross-linking of proteins that can lead to the masking or destruction of antigen sites and, subsequently, poor antibody staining As used herein, “antigen retrieval” or “antigen unmasking” refers to methods for increasing antigen accessibility or recovering antigenicity in, for example, formalin-fixed, paraffin-embedded tissue samples. Any method for making antigens more accessible for antibody binding may be used in the practice of the invention, including those antigen retrieval methods known in the art. See, for example, Hanausek and Walaszek, eds. (1998) Tumor Marker Protocols (Humana Press, Inc., Totowa, N.J.) and Shi et al., eds. (2000) Antigen Retrieval Techniques: Immunohistochemistry and Molecular Morphology (Eaton Publishing, Natick, Mass.).

Antigen retrieval methods include but are not limited to treatment with proteolytic enzymes (e.g., trypsin, chymotrypsin, pepsin, pronase, and the like) or antigen retrieval solutions. Antigen retrieval solutions of interest include, for example, citrate buffer, pH 6.0, Tris buffer, pH 9.5, EDTA, pH 8.0, L.A.B. (“Liberate Antibody Binding Solution,” Polysciences, Warrington, Pa.), antigen retrieval Glyca solution (Biogenex, San Ramon, Calif.), citrate buffer solution, pH 4.0, Dawn® detergent (Proctor & Gamble, Cincinnati, Ohio), deionized water, and 2% glacial acetic acid. In some embodiments, antigen retrieval comprises applying the antigen retrieval solution to a formalin-fixed tissue sample and then heating the sample in an oven (e.g., at 60° C.), steamer (e.g., at 95° C.), or pressure cooker (e.g., at 120° C.) at specified temperatures for defined time periods. In other aspects of the invention, antigen retrieval may be performed at room temperature. Incubation times will vary with the particular antigen retrieval solution selected and with the incubation temperature. For example, an antigen retrieval solution may be applied to a sample for as little as 5, 10, 20, or 30 minutes or up to overnight. The design of assays to determine the appropriate antigen retrieval solution and optimal incubation times and temperatures is standard and well within the routine capabilities of those of ordinary skill in the art.

Following antigen retrieval, samples are blocked using an appropriate blocking agent (e.g., hydrogen peroxide). An antibody directed to a biomarker of interest is then incubated with the sample for a time sufficient to permit antigen-antibody binding. In particular embodiments, at least five antibodies directed to five distinct biomarkers are used to evaluate the prognosis of a breast cancer patient. Where more than one antibody is used, these antibodies may be added to a single sample sequentially as individual antibody reagents, or simultaneously as an antibody cocktail. Alternatively, each individual antibody may be added to a separate tissue section from a single patient sample, and the resulting data pooled.

Techniques for detecting antibody binding are well known in the art. Antibody binding to a biomarker of interest can be detected through the use of chemical reagents that generate a detectable signal that corresponds to the level of antibody binding, and, accordingly, to the level of biomarker protein expression. For example, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell or tissue staining that corresponds to expression level of the biomarker of interest. Enzymes of particular interest include horseradish peroxidase (HRP) and alkaline phosphatase (AP). Commercial antibody detection systems, such as, for example the Dako Envision+system (Glostrup, Denmark) and Biocare Medical's Mach 3 system (Concord, Calif.), can be used to practice the present invention.

The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody. The antibodies used to practice the invention are selected to have specificity for the biomarker proteins of interest. Methods for making antibodies and for selecting appropriate antibodies are known in the art. See, for example, Celis, ed. (2006) Cell Biology: A Laboratory Handbook, 3rd edition (Elsevier Academic Press, New York). In some embodiments, commercial antibodies directed to specific biomarker proteins can be used to practice the invention. The antibodies of the invention can be selected on the basis of desirable staining of histological samples. That is, the antibodies are selected with the end sample type (e.g., formalin-fixed, paraffin-embedded breast tumor tissue samples) in mind and for binding specificity.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride, and phycoerythrin. An example of a luminescent material is luminol. Examples of bioluminescent materials include luciferase, luciferin and aequorin. Examples of suitable radioactive materials include ¹²⁵I, ¹³¹I, ³⁵S, and ³H.

In regard to detection of antibody staining in the immunohistochemistry methods of the invention, there also exist in the art, video-microscopy and software methods for the quantitative determination of an amount of multiple molecular species (e.g., biomarker proteins) in a biological sample where each molecular species present is indicated by a representative dye marker having a specific color. Such methods are also known in the art as a colorimetric analysis methods. In these methods, video-microscopy is used to provide an image of the biological sample after it has been stained to visually indicate the presence of a particular biomarker of interest. See, for example, U.S. Pat. Nos. 7,065,236 and 7,133,547, which disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

### Proteomics

The term “proteome” is defined as the totality of the proteins present in a sample (e.g., tissue, organism or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE) or liquid/gas chromatography; (2) identification of the individual proteins recovered from the gel or contained within a column fraction, for example, by mass spectrometry or N-terminal sequencing, and (3) analysis of the data using bioinformatics. Proteomics methods are valuable supplements to other methods of gene expression profiling, and can be used, alone or in combination with other methods, to detect the products of the biomarkers of the present invention.

### Kits

Kits for practicing the methods of the invention are further provided. By “kit” is intended any manufacture (e.g., a package or a container) including at least one reagent, such as a nucleic acid probe, an antibody or the like, for specifically detecting the expression of a biomarker of the invention. The kits can be promoted, distributed or sold as units for performing the methods of the present invention. Additionally, kits can contain a package insert describing the kit and methods for its use.

In particular embodiments, kits for diagnosing and for evaluating the prognosis of a breast cancer patient including detecting biomarker overexpression at the nucleic acid level are provided. Such kits are compatible with both manual and automated nucleic acid detection techniques (e.g., gene arrays). These kits include, for example, at least five nucleic acid probes that specifically bind to five distinct biomarker nucleic acids or fragments thereof.

In other embodiments, kits for practicing the immunohistochemistry methods of the invention are provided. Such kits are compatible with both manual and automated immunohistochemistry techniques (e.g., cell staining). These kits include at least five antibodies for specifically detecting the expression of at least five distinct biomarkers. Each antibody can be provided in the kit as an individual reagent or, alternatively, as an antibody cocktail comprising at least five antibodies directed to at least five different biomarkers.

Any or all of the kit reagents can be provided within containers that protect them from the external environment, such as in sealed containers. Positive and/or negative controls can be included in the kits to validate the activity and correct usage of reagents employed in accordance with the invention. Controls can include samples, such as tissue sections, cells fixed on glass slides, RNA preparations from tissues or cell lines, and the like, known to be either positive or negative for the presence of at least five different biomarkers. The design and use of controls is standard and well within the routine capabilities of those of ordinary skill in the art.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation:

### EXPERIMENTAL

### Methods

### Tissue Samples, RNA Preparations and Microarray Protocols

One hundred forty-six patients representing all disease stages and grades, represented by 162 breast tumor specimens (with 23 repeated or paired samples) and 10 normal breast tissue samples (giving 195 total arrays) were used for expression profiling. Most of these samples have been described (Weigelt et al., Cancer Res. 65:9155-58, 2005; Hu et al., BMC Genomics 7:96, 2006; Oh et al., J. Clin. Oncol. 24:1656-64, 2006), with 39 being new to this study, and all of which were collected using IRB approved protocols. In addition, 3 additional normal breast and 4 normal liver samples were taken from 6 autopsy patients and used for analyses focused on sample handling-associated profiles. In total, 134 primary tumor specimens, 8 regional metastases and 18 distant metastasis specimens were assayed. For the distant metastasis samples, no actual bone marrow metastasis samples were assayed, however, at least 6 of the patients with distant disease were noted as having metastases in the bone. Patients were heterogeneously treated in accordance with the standard of care dictated by their disease stage, ER and HER-2 status. Most primary tumor and regional metastasis samples (except 4 primary tumors) were collected at the time of first surgery, however, 17 of 18 distant metastasis samples were obtained from patients who had received prior treatment and 10 of 18 were obtained from autopsy patients.

Total RNA isolation and microarray protocols are described in Hu et al. (Biotechniques 38:121-24, 2005). The total RNA labeling and hybridization protocol used was the Agilent (Santa Clara, Calif.) low RNA input linear amplification kit. Each sample was assayed versus a common reference sample that was a mixture of Stratagene's (La Jolla, Calif.) Human Universal Reference total RNA (Novoradovskaya et al., BMC Genomics 5:20, 2004) (100 μg) enriched with equal amounts of RNA (0.3 μg each) from MCF7 and ME16C cell lines. Microarray hybridizations were carried out on Agilent Human 22,000 feature oligonucleotide microarrays (1A-v1,1A-v2 and custom designed 1A-v1 based microarrays) using 2 μg of Cy3-labeled Reference and 2 μg of Cy5-labeled experimental sample. All microarrays were scanned using an Axon Scanner GenePix 4000B, analyzed with GenePix Pro 4.1 (Molecular Devices, Sunnyvale, Calif.) and loaded into the University of North Carolina (UNC) Microarray Database where a Lowess normalization procedure was performed. All microarray data associated with this study have been deposited into the Gene Expression Omnibus under accession number GSE3521.

### Supervised Microarray Data Analysis

The background subtracted, Lowess normalized log₂ ratio of Cy5 over Cy3 intensity values were first filtered to select genes that had a signal intensity of at least 30 units above background in both the Cy5 and Cy3 channels. Only genes that met these criteria in at least 70% of the 195 microarrays were included for subsequent analysis. Next, each patient was classified according to the following “MetScore” criteria. MetScore 1: patients had a primary tumor and were clinically node negative (N=0) and distant metastasis negative (M=0); MetScore 2: patients with a primary tumor and a regional metastasis (N=1-3) and no distant metastasis (M=0); MetScore 3: patients with confirmed distant disease at the time of diagnosis (M=1 and any N) or were represented by an actual distant metastasis sample. Thus, no knowledge of relapse rates or overall survival was used for any MetScore-based microarray analysis. For supervised analysis purposes, if a patient had a primary tumor and a regional metastasis sample both assayed on microarrays, both were classified as MetScore 2, and if a patient had a primary, regional and/or distant metastasis sample, all were classified as MetScore 3. A multi-class significance analysis of microarrays (SAM) analysis using a single sample from each patient was performed, biasing the sample selection to use the regional metastasis sample for MetScore 2 patients, and the distant metastasis sample for MetScore 3 patients (146 arrays, see Supplemental Table 1 for the actual samples used). For the SAM, 10 nearest neighbors were used for the missing data imputation, and the gene set that was associated with the MetScore 1-2-3 distinction, and which gave 1,195 genes at a False Discovery Rate (FDR) of 5% (59 potential false-positive genes), was identified. This gene set was used to perform a one-way average linkage hierarchical cluster analysis using the program “Cluster” (Eisen et al., Proc. Natl. Acad. Sci. USA 95:14863-68, 1998), with the data being displayed relative to the median expression for each gene using “Java Treeview” (Saldanha, Bioinformatics 20:3246-48, 2004).

### Cross Validation Analyses

Relationships between the gene expression data and the MetScore classification were further examined using a 10-fold cross-validation (CV) analysis to identify a set of genes that might distinguish a MetScore group from the others, and to determine how accurate this classification might be. Ten-fold CV using five different statistical predictors including PAM (Tibshirani et al., Proc. Natl. Acad. Sci. USA 99:6567-72, 2002), a k-Nearest Neighbor Classifier (KNN) with either Euclidean distance or one-minus-Spearman-correlation as the distance function and a Class Nearest Centroid (CNC) metric with either Euclidean distance or one-minus-Spearman-correlation as the distance function, were used as described in Chung et al. (Cancer Cell 5:489-500, 2004). Ten-fold CV was performed using the five different statistical predictors with the reported CV prediction accuracies being the average of the five predictors (Table 1).

TABLE 1
10-fold CV prediction accuracies of MetScore categories relative to each
other

| MetScores | Spearman | Euclidean | Spearman- | | | average |
|---|---|---|---|---|---|---|
| Compared | nearest centroid | nearest centroid | KNN | Euclidean-KNN | PAM | prediction accuracy |
| 1 vs. 2 | 0.56 | 0.61 | 0.61 | 0.65 | 0.63 | 0.61 |
| 1 vs. 3 | 0.82 | 0.84 | 0.85 | 0.83 | 0.80 | 0.83 |
| 2 vs. 3 | 0.83 | 0.82 | 0.81 | 0.82 | 0.81 | 0.82 |

### Outcome and ANOVA Analyses

Training set patients were assigned a MetScore and analyzed by Univariate Kaplan-Meier analysis using a log-rank test as performed using WinSTAT for excel (R. Fitch Software, Lehigh Valley, Pa.). In addition, each sample was assigned an “intrinsic subtype” as described in Fan et al. (N. Engl. J. Med. 355:560-69, 2006), where a Centroid was created for each of the following intrinsic subtypes: Basal-like, Luminal A, Luminal B, HER2+/ER−, and Normal-like.

For the VEGF-profile, an average expression value across all 13-genes (RRAGD, FABP5, UCHL1, GAL, PLOD, DDIT4, VEGF, ADM, ANGPTL4, NDRG1, NP, SLC16A3, and C14ORF58) was determined and the patients were placed into a three group classification based their 13-gene average log₂ expression ratio and using the cut off values (−0.01 and 0.98) that were identified using X-tile (Camp et al., Clin. Cancer Res. 10:7252-59, 2004). Analyses using the VEGF-profile and the training set cutoffs were also applied to an independent test set of 295 patients assayed on Agilent microarrays (i.e., NKI295; Chang et al., Proc. Natl. Acad. Sci. USA 102:3738-43, 2005), and on another test set of patients assayed on Affymetrix microarrays (Pawitan et al., Cancer Res. 7: 953-64, 2005). To perform these across data set analyses, for the NKI295 dataset the log ratio of red channel intensity versus green channel intensity was used and the data was median centered for every gene across the 295 arrays. The NKI295 dataset was next Distance Weighted Discrimination (DWD) normalized (Benito et al., Bioinformatics 20:105-14, 2004) with the UNC training dataset after collapsing by NCBI Entrez GeneID. After DWD normalization, the NKI295 data was also column standardized. For the Affymetrix dataset the probe level intensity CEL files were processed by Robust Multi-chip Average (RMA). The probe sets log intensity was median centered for every gene across all the arrays. The Affymetrix dataset was also DWD normalized relative to the UNC training data after collapsing by NCBI Entrez GeneID, and was column standardized.

For the evaluation of the autopsy samples specifically, the MetScore classification system was modified into the following six categories where the autopsy patients were removed from the MetScore 3 group and placed into their own group. Group 1: MetScore 1 patients; Group 2: MetScore 2 patients; Group 3: MetScore 3 patients with all true distant metastasis samples removed; Group 4: autopsy patient distant metastasis samples (6 total); Group 5: distant metastasis samples that were not autopsy patients; and Group 6: normal tissues from autopsy patients. Each patient was evaluated for three different profiles, the 13-gene VEGF signature and two prostate radical prostatectomy sample handling-associated signatures (Dash et al., Am. J. Pathol. 161:1743-48, 2002; Lin et al., J. Clin. Oncol. 24:3763-70, 2006), which were chosen as being representative of solid tumor sample handling issues. As many genes as possible were taken for each signature, and an average value for each gene set for each patient was calculated. Next, Chi-squared and ANOVA analyses were performed using SAS (Cary, N.C.) software (version 9.1) to determine if a statistically significant correlation existed between the six groups and a given profile.

Multivariate analysis of the NKI295 test set using Cox proportional hazards modeling was conducted in SAS version 9.1. A Cox hazard model (Tables 2A-C) that included estrogen receptor status (binary variable coded as positive versus negative), tumor size (binary variable coded as ≦2 cm versus >2 cm), lymph node status (indicator coding with three categories: 0, 1-3, >3 positive nodes or M=1), age (continuous variable, formatted in decades), grade (coding as grade 1 versus 2, and grade 1 versus 3), and treatment (binary variable coded as yes if treatment with chemo and/or hormonal therapy, no if no adjuvant therapy was given), and the VEGF-profile of low, intermediate or high as a single categorical variable, was tested. Another model was also tested that included all the clinical variables, the VEGF-profile, the NKI 70-gene profile, a microarray-based version of the Genomic Health Recurrence Score, the Wound Response profile, the Intrinsic Subtypes profiles as described and taken from Fan et al. (N. Engl. J. Med. 355:560-69, 2006), an estrogen-regulated IE-IIE profile (Oh et al., J. Clin. Oncol. 24:1656-64, 2006), and a p53-mutation profile (Troester et al., BMC Cancer 6:276, 2006).

TABLE 2
Cox proportional hazards models for overall survival using the NKI295
patient test data set

| | | | | | | | |
|---|---|---|---|---|---|---|---|
| | | | | | | | 95% Hazard |
| | | | | | | | Ratio |
| | | | Standard | Chi- | Pr > | Hazard | Confidence |
| Variable | DF | Estimate | Error | Square | ChiSq | Ratio | Limits |

A. Model containing the clinical variables and the VEGF-profile

| | | | | | | | | |
|---|---|---|---|---|---|---|---|---|
| Age | 1 | −0.04752 | 0.01975 | 5.7917 | 0.0161 | 0.954 | 0.917 | 0.991 |
| ER | 1 | −0.46767 | 0.27578 | 2.8759 | 0.0899 | 0.626 | 0.365 | 1.076 |
| Grade2vs1 | 1 | 1.40563 | 0.54303 | 6.7003 | 0.0096 | 4.078 | 1.407 | 11.822 |
| Grade3vs1 | 1 | 1.69868 | 0.54146 | 9.8421 | 0.0017 | 5.467 | 1.892 | 15.799 |
| Tumor size | 1 | 0.37356 | 0.24214 | 2.3801 | 0.1229 | 1.453 | 0.904 | 2.335 |
| Node | 1 | 0.23801 | 0.21814 | 1.1906 | 0.2752 | 1.269 | 0.827 | 1.946 |
| Treatment | 1 | −0.29834 | 0.33765 | 0.7807 | 0.3769 | 0.742 | 0.383 | 1.438 |
| VEGF-profile | 1 | 0.52638 | 0.19248 | 7.4791 | 0.0062 | 1.693 | 1.161 | 2.469 |

B. Model containing the clinical variables and multiple gene expression
profiles

| | | | | | | | | |
|---|---|---|---|---|---|---|---|---|
| Age | 1 | −0.04619 | 0.02082 | 4.9239 | 0.0265 | 0.955 | 0.917 | 0.995 |
| ER | 1 | −0.67876 | 0.40687 | 2.783 | 0.0953 | 0.507 | 0.229 | 1.126 |
| Grade2vs1 | 1 | 0.59814 | 0.56923 | 1.1041 | 0.2934 | 1.819 | 0.596 | 5.55 |
| Grade3vs1 | 1 | 0.75132 | 0.58444 | 1.6526 | 0.1986 | 2.12 | 0.674 | 6.665 |
| Tumorsize | 1 | 0.52004 | 0.25062 | 4.3055 | 0.038 | 1.682 | 1.029 | 2.749 |
| Node | 1 | 0.14236 | 0.23399 | 0.3702 | 0.5429 | 1.153 | 0.729 | 1.824 |
| Treatment | 1 | −0.27803 | 0.35993 | 0.5967 | 0.4398 | 0.757 | 0.374 | 1.533 |
| VEGF-profile | 1 | 0.5546 | 0.20885 | 7.0515 | 0.0079 | 1.741 | 1.156 | 2.622 |
| GHI RS | 1 | 0.43908 | 0.34966 | 1.5768 | 0.2092 | 1.551 | 0.782 | 3.078 |
| 70-gene | 1 | 0.9354 | 0.49524 | 3.5675 | 0.0589 | 2.548 | 0.965 | 6.726 |
| Wound Response | 1 | 0.78386 | 0.50588 | 2.4009 | 0.1213 | 2.19 | 0.812 | 5.902 |
| LumA-vs-LumB | 1 | −0.02133 | 0.4673 | 0.0021 | 0.9636 | 0.979 | 0.392 | 2.446 |
| LumA-vs-Basal | 1 | −1.0631 | 0.58521 | 3.3001 | 0.0693 | 0.345 | 0.11 | 1.087 |
| LumA-vs- | 1 | −0.60342 | 0.55221 | 1.1941 | 0.2745 | 0.547 | 0.185 | 1.614 |
| HER2+/ER− | | | | | | | | |
| LumA-vs-Normal | 1 | −0.09803 | 0.53337 | 0.0338 | 0.8542 | 0.907 | 0.319 | 2.579 |
| Estrogen IE-vs-IIE | 1 | 0.38071 | 0.43547 | 0.7643 | 0.382 | 1.463 | 0.623 | 3.436 |
| P53-mutant-profile | 1 | 0.03252 | 0.39487 | 0.0068 | 0.9344 | 1.033 | 0.476 | 2.24 |

C. Backwards selected model from Table 2B showing the final parameters

| | | | | | | | | |
|---|---|---|---|---|---|---|---|---|
| Age | 1 | −0.04872 | 0.01955 | 6.2084 | 0.0127 | 0.952 | 0.917 | 0.99 |
| Tumor size | 1 | 0.51498 | 0.23738 | 4.7066 | 0.03 | 1.674 | 1.051 | 2.665 |
| VEGF-profile | 1 | 0.52533 | 0.17544 | 8.9659 | 0.0028 | 1.691 | 1.199 | 2.385 |
| GHI RS | 1 | 0.66503 | 0.30066 | 4.8926 | 0.027 | 1.945 | 1.079 | 3.505 |
| 70-gene | 1 | 1.24128 | 0.44705 | 7.7096 | 0.0055 | 3.46 | 1.441 | 8.31 |

Associations between a tumor's intrinsic subtype, the 13-gene VEGF-profile and other published expression profiles implicated in metastasis biology that included: A) the 70-gene outcome predictor developed by van't Veer et al. (N. Engl. J. Med. 347:1999-2009, 2002; Nature 415:530-36, 2002); B) the “wound-response” profile (Chang et al., PLoS Biol. 2:E7, 2004); C) the hypoxia-induced cell line signature (Chi et al., PLoS Med. 3:e47, 2006); D) the 11-gene BMI/stem cell signature (Glinsky et al., J. Clin. Invest. 115:1503-21, 2005); E) a bone metastasis signature (Kang et al., Cancer Cell 3:537-49, 2003); F) a lung metastasis signature (Minn et al., Nature 436:518-24, 2005); and G) the expression profiles of HIF1a, Snail (Moody et al., Cancer Cell 8:197-209, 2005) and Twist (Yang et al., Cell 117:927-39, 2004) were also tested for. As many genes as was possible were extracted from the microarrays for each predictor and the classification scheme described by the authors was followed. For the bone metastasis and lung metastasis signatures, an average value for each patient using the 43 genes that were highly expressed in the cell line derivatives that metastasized to the bone/lung were created. For the 11-gene stem cell signature, an average value across all 11-genes was created. A “glycolysis-profile” was also created by starting with the 9 glycolysis genes/probes present on the array, then filtering for probes that showed >30 intensity units in both channels and then selecting for 70% good data across all samples. The subset of glycolysis gene probes that passed filtering and showed a Pearson correlation of greater than 0.4 were selected, resulting in the selection of 6 of 9 glycolysis genes, GPI (glucose phosphate isomerase), PKM2 (pyruvate kinase, muscle), PFKP (phosphofructokinase, platelet), PGK1 (phosphoglycerate kinase 1), GAPD (glyceraldehyde-3-phosphate dehydrogenase), and ENO1 (enolase 1, alpha), which were then used to create an average profile for each patient.

Correlations between profiles using multiple methods (Table 3) were then examined. For quantized profile testing, Chi-squared analysis and Fischer's exact T-test were used. For continuous variable testing, ANOVA analyses were performed. Finally, a calculation of the Cramer's V statistic for the evaluation of the strength of association between two quantized variables was also performed (see, Oh et al., J. Clin. Oncol. 24:1656-64, 2006).

TABLE 3
Correlation analysis of multiple gene expression profiles linked
to metastasis biology or formation compared to each other
Quantized Variables Testing

| | | Chi-square P- | | Fisher Exact |
|---|---|---|---|---|
| Primary Signature | Test Signature | value | Cramer's V | P-value |
| VEGF-profile | MetScore | 0.0002 | 0.272 | 4.80E−04 |
| VEGF-profile | NKI 70-gene profile | 0.0008 | 0.3126 | 3.60E−04 |
| VEGF-profile | Wound Response Profile | 0.0001 | 0.3524 | 3.78E−06 |
| VEGF-profile | Intrinsic Subtype | <.0001 | 0.4223 | 4.29E−11 |
| VEGF-profile | Cell line hypoxia-profile | <.0001 | 0.6394 | 1.10E−15 |
| Intrinsic Subtype | MetScore | 0.0054 | 0.2578 | 7.09E−04 |
| Intrinsic Subtype | Cell line hypoxia-profile | <.0001 | 0.739 | 1.40E−20 |
| Intrinsic Subtype | VEGF-profile | <.0001 | 0.4223 | 4.29E−11 |
| Intrinsic Subtype | NKI 70-gene profile | <.0001 | 0.4449 | 5.94E−06 |
| Intrinsic Subtype | Wound Response Profile | <.0001 | 0.7389 | 1.56E−16 |

Continuous Variables Testing

| Primary Signature | Test Signature | ANOVA P-value |
|---|---|---|
| VEGF-profile | Bone Metastasis profile | <.0001 |
| VEGF-profile | Lung Metastasis profile | <.0001 |
| VEGF-profile | Snail1 | <.0001 |
| VEGF-profile | Twist1 | 0.3 |
| VEGF-profile | 11 gene stem cell profile | 0.0074 |
| VEGF-profile | Glycolysis-Profile | <.0001 |
| VEGF-profile | Fibroblast-profile | 0.7 |
| VEGF-profile | HIF1α | 0.0004 |
| Intrinsic Subtype | Bone Metastasis profile | 0.054 |
| Intrinsic Subtype | Lung Metastasis profile | 0.036 |
| Intrinsic Subtype | Snail1 | 0.0002 |
| Intrinsic Subtype | Twist1 | 0.2 |
| Intrinsic Subtype | 11 gene stem cell profile | <.0001 |
| Intrinsic Subtype | Glycolysis-Profile | <.0001 |
| Intrinsic Subtype | Fibroblast-profile | 0.012 |
| Intrinsic Subtype | HIF1α | 0.0033 |

### In Situ Hybridization

In situ hybridization (ISH) on Tissue Microarray (TMA) sections containing 250 different human breast tumors (not related to the 146 used for microarray analysis) was performed as described by West et al. (Am. J. Pathol. 165:107-13, 2004). Briefly, digoxigenin (DIG)-labeled sense and anti-sense RNA probes were generated by PCR amplification of approximately 450 by products with the T7 promoter incorporated into the primers; the primer sequences used for amplification were VEGF (Forward-TCTCCCTGATCGGTGACAGT (SEQ ID NO:1); Reverse-TCGAAAAACTGCACTA GAGACAA (SEQ ID NO:2)), ANGPTL4 (Forward-GGGAATCTTCTGGAAGACCTG (SEQ ID NO:3); Reverse-TACACACAACAGCACCAGCA (SEQ ID NO:4)) and ADM (Forward-GTGTTTGCCAGGCTTAAGGA (SEQ ID NO:5); Reverse-TCGGTGTTT CCTTCTTCCAC (SEQ ID NO:6)). In vitro transcription was performed with a DIG RNA-labeling kit and T7 polymerase according to the manufacturer's protocol (Roche Diagnostics, Indianapolis, Ind.).

### Expression Patterns Associated With Primary Tumors Versus Metastases

To identify gene expression changes occurring during tumor progression from localized to a regional metastasis and ultimately to a distant metastasis, 195 microarrays from 146 patients were performed, representing 134 primary tumor specimens, 9 regional metastases and 19 distant metastasis specimens. Each patient was classified according to the MetScore criteria described herein. For this analysis, if the patient's primary tumor and metastasis sample were assayed, both were categorized into the MetScore 2 (if regional) or MetScore 3 (if distant metastasis) categories, which was based upon previous findings that primary tumors and their associated metastases are similar (Perou et al., Nature 406:747-52, 2000; Weigelt et al., Cancer Res. 65:9155-58, 2005). This scoring system was highly predictive of patient outcomes.

Using the MetScore classifications, CV analyses was performed to determine if any MetScore group might be distinct relative to the others. No gene set was identified that showed a clear and stereotyped expression progression across the MetScore groups, however, there were differences in the MetScore 3 samples that distinguished them from the other two categories. The most notable changes included the low expression of the fibroblast/mesenchymal gene set (and a corresponding lack of fibroblasts as defined by histological examination) and the high expression of the 13-gene VEGF-profile. Low accuracy rates (56-65%) for the prediction of MetScore 1 versus MetScore 2 specimens were observed. However, when MetScore 1 versus MetScore 3 samples (80-85%) or MetScore 2 versus MetScore 3 samples (81-83%) were compared, high accuracy rates were obtained (Table 1). The VEGF-profile represents a compact in vivo defined gene expression program that includes a combination of cell intrinsic and cell extrinsic factors that likely allow tumors that possess it to be better adapted to life under oxygen-poor conditions

A multi-class SAM analysis (Tusher et al., Proc. Natl. Acad. Sci. USA 98:5116-21, 2001) using a single sample from each of the 146 patients was performed, and a 1,195-gene set at a 5% FDR was obtained. This gene set was then used in a one-way average linkage hierarchical clustering analysis where the samples were first ordered according to MetScore status, and then according to their correlation to the average profile (i.e., centroid) of the MetScore 3 class. Clinical node status, distant metastasis status, estrogen and progesterone status, and intrinsic subtype were determined. This analysis demonstrated that some MetScore 1 and 2 samples actually had a MetScore 3 profile (see, also, Ramaswamy et al., Nat. Genet. 33:49-54, 2003).

The gene expression patterns from the SAM analysis were complex and there were few, if any, that directly correlated with a simple progression from MetScore 1 to 2 to 3. Included within this gene set were many clusters/gene sets that have been identified previously, including a luminal/ER+ expression pattern (van't Veer et al., Nature 415:530-36, 2002; Gruvberger et al., Cancer Res. 61:5979-84, 2001; Hoch et al., Int. J. Cancer 84:122-28, 1999) and a proliferation signature (Perou et al., Nature 406:747-52, 2000; Whitfield et al., Mol. Biol. Cell 13:1977-2000, 2002), both of which are integral parts of a gene expression based assay that predicts the likelihood of recurrence in ER+ and tamoxifen-treated patients (Paik et al., N. Engl. J. Med. 351:2817-26, 2004). In addition, many other biologically important gene sets were identified including an immediate early gene cluster containing c-FOS, JUNB and some of their known target genes (Iyer et al., Science 283:83-87, 1999), a set of fibroblast/ECM genes containing PLAU, THSB2 and multiple Collagen genes that was low in most MetScore 3 samples, a set of immune cell genes, and a novel gene set containing CXCL12. CXCL12 was the top ranked gene from the SAM analysis and has been identified as a chemokine whose high expression promotes tumor cell proliferation, migration and invasion (Allinen et al., Cancer Cell 6:17-32, 2004). Analysis of these individual clusters/gene sets by EASE (Hosack et al., Genome Biol. 4:R70, 2003) identified many significant Gene Ontology categories that included transcription regulation and DNA/nucleic acid binding for the FOS-JUN cluster, while the fibroblast/ECM cluster was over represented for extracellular matrix, cell adhesion and communication, organogenesis, development, and regulation of protease activity. The CXCL12 cluster was over represented for cell adhesion, cell migration and extracellular matrix. A small but distinct 13-gene profile, containing VEGF, ADM, ANGPTL4, RRAGD, FABP5, UCHL1, GAL, PLOD, DDIT4, NDRG1, NP, SLC16A3, and C14ORF58 was identified, as discussed in greater detail below.

### Associations Between MetScore, Tumor Intrinsic Subtypes And Outcomes

Previous work identified at least five major “intrinsic” subtypes of breast cancer that are of prognostic and predictive value, namely Luminal A, Luminal B, Basal-like, HER2+/ER− and Normal-like (Perou et al., Nature 406:747-52, 2000; Hu et al., BMC Genomics 7:96, 2006; Sorlie et al., Proc. Natl. Acad. Sci. USA 100:8418-23, 2003). Subtype classification of the tumors using the Intrinsic/UNC list and the centroid predictor described in Fan et al. (N. Engl. J. Med. 355:560-69, 2006) showed statistically significant outcome predictions on the training data set. A Chi-squared test (p=0.0006) showed that intrinsic subtype was significantly correlated with MetScore, with the Basal-like and HER2+/ER− groups being the most frequent in the MetScore 3 category, and with no Luminal A samples being in the MetScore 3 group. Correlations between tumor subtype, node status and disease stage have been recently described (Calza et al., Breast Cancer Res. 8:R34, 2006; Carey et al., Jama 295:2492-502, 2006), and were recapitulated here.

### Analysis Of The 13-Gene VEGF-Profile

A small but distinct cluster of genes containing VEGF was identified that showed high expression in MetScore 3 tumors relative to MetScore 1 and 2 tumors. This gene cluster contained several secreted proteins that have been implicated in endothelial cell (VEGF and ANGPTL4), lymphatic cell (ADM) and smooth muscle cell dynamics (GAL). As a first step in evaluating this profile, ISH was performed to determine what cell type was producing VEGF, ANGPTL4 and ADM. In the vast majority of cases that showed strong ISH positivity (which totaled approximately 10% of the 250 tumors tested), it was the tumor cells themselves that produced the mRNA for these three genes, and typically all three were produced. In a few cases both tumor and fibroblasts showed ISH positivity, but this was rare.

As a second step in the evaluation of the VEGF-profile, an average expression ratio for each patient across all 13-genes was created and correlations with outcome were examined. By dividing the patients into low, intermediate and high expression groups using cutoffs determined by X-tile (Camp et al., Clin. Cancer Res. 10:7252-59, 2004), it was determined that the VEGF-profile was prognostic of relapse-free (RFS) and overall survival (OS), with high expression portending a poor outcome. Applying the VEGF-profile classification rules to an independent test set of 295 patients (i.e., NKI295; van de Vijver et al., N. Engl. J. Med. 347:1999-2009, 2002; Chang et al., Proc. Natl. Acad. Sci. USA 102:3738-43, 2005) also significantly predicted outcomes. This classification rule was also of prognostic value on a second test set of patients assayed on Affymetrix microarray (Pawitan et al., Cancer Res. 7: 953-64, 2005).

A multivariate Cox proportional hazards analysis on the NKI295 test set using overall survival was performed using clinical variables and the VEGF-profile, and it was determined that the VEGF-profile was a significant predictor of outcomes (Table 2A). In Fan et al. (N. Engl. J. Med. 355:560-69, 2006), prognostic powers and concordance across multiple expression predictors, including the intrinsic subtypes, the NKI 70-gene signature, a microarray-based version of the Genomic Health Recurrence Score, and the wound-response profile using this same NKI295 patient data set, were evaluated. Other profiles of prognostic significance, including a profile based upon estrogen-regulated genes (Oh et al., J. Clin. Oncol. 24:1656-64, 2006) and p53 mutation status (Troester et al., BMC Cancer 6:276, 2006) have also been identified. Therefore, a Cox proportional hazards analysis (Table 2B) and backwards variable selection (Table 2C) was performed to evaluate a model that contained all of the aforementioned gene expression predictors and the clinical variables. The final model contained both clinical parameters (age and tumor size) and multiple gene expression predictors including the VEGF-profile (Table 2C). Similar results were also obtained when performing Cox proportional hazards analyses using the endpoint of time to first relapse of any kind, or time to distant metastasis formation.

### Analysis Of A Glycolysis-Profile And HIF1α Gene Expression

A biological implication of the VEGF-profile is that it may be related to a tumor's response to hypoxic conditions and/or high growth rates, which historically has been referred to as the Warburg effect (Warburg, Science 124:269-70, 1956; Semenza et al., Novartis Found. Symp. 240:251-60; discussion 60-64, 2001). A central tenant of the Warburg effect is that a tumor's metabolism becomes more dependent upon glycolysis due to hypoxic conditions. To examine glycolysis in tumors using a genomic approach, a “glycolysis-profile” was created, using the six most highly correlated glycolysis gene probes (GPI, PKM2, PFKP, PGK1, GAPD, and ENO1). The 13-gene VEGF-profile and the glycolysis-profile are correlated, which is supported by an ANOVA (p<0.001, Table 3).

As a known regulator of VEGF expression, it was determined that HIF1α (hypoxia-inducible factor 1, alpha subunit) gene expression was correlated with expression of the 13-gene VEGF-profile (p=0.0003, Table 3). The promoter region of each of the genes in the VEGF-profile was examined using the program rVISTA (Loots et al., Genome Res. 12:832-39, 2002), and it was determined that DDIT4, VEGF, NDRG1, SLC16A3, PLOD, ADM, ANGPTL4, and C14ORF58 have potential hypoxia-response elements within 2000 by upstream of their start codons. It is already known that many of these genes, including VEGF (Fang et al., Cancer Res. 61:5731-35, 2001), ADM (Frede et al., Cancer Res. 65:4690-97, 2005) and DDIT4 (Schwarzer et al., Oncogene 24:1138-49, 2005) are HIF1α-regulated. The gene PH-4 (hypoxia-inducible factor prolyl 4-hydroxylase), which is the gene/protein needed to inactivate HIF1α via prolyl-hydroxylation, was anti-correlated in expression relative to HIF1α and the 13-gene VEGF-signature. Nearly identical results were also observed on the test set of NKI295 patients.

### Sample Handling-Associated Signatures

It has previously been shown that there are gene expression patterns associated with prolonged sample handling times. Therefore, the autopsy tumor samples were separated from the other MetScore 3 samples and evaluated for their expression of the VEGF-profile and two previously published epithelial tumor sample handling-associated signatures (Dash et al., Am. J. Pathol. 161:1743-48, 2002; Lin et al., J. Clin. Oncol. 24:3763-70, 2006). For these analyses, a modified MetScore classification system was used where the MetScore 1 and 2 groups remained the same, but the MetScore 3 group was broken into three groups that were MetScore 3 patients represented by primary tumors or a regional metastasis (11 total), autopsy patient tumors (6 total) and then the remaining distant metastasis samples (9 total). In addition, a group was also created using 7 normal tissue samples taken from the 6 autopsy patients. The results using this modified MetScore classification system and ANOVA analyses showed a statistically significant association between the average expression of the 13-gene VEGF profile and these six groups, with the VEGF-profile being the highest in the two autopsy patient containing groups. However, when the data for the 13-genes was displayed in heat map format and the sample order was maintained according to the six class distinction, only part of the 13-gene VEGF profile, that is, VEGF, ADM and ANGPL4, were high in the autopsy normals, while the complete signature tended to be high in the autopsy tumor and many MetScore 3 patients.

When the two previously published sample handling-associated profiles were tested, the profile of Lin et al. (J. Clin. Oncol. 24:3763-70, 2006) showed the highest expression in the autopsy normal samples, but was lowest in the autopsy tumors and remaining MetScore 3 patients. The profile of Dash et al. (Am. J. Pathol. 161:1743-48, 2002) showed the highest expression in the autopsy normal tissue group, lowest in the remaining MetScore 3 patients and low-moderate expression in the autopsy tumor samples. In addition, neither of the sample handling-associated profiles were able to predict outcomes in the training or NKI295 data set. Thus, the previously defined prolonged sample handling-associated profiles were present in the autopsy normal samples, but not in the autopsy tumor samples nor in the remaining MetScore 3 patients.

### Fibroblast Signature

To examine the potential fibroblast/mesenchymal cell amounts present within each MetScore group, each patients average expression value of the genes contained within the fibroblast/ECM gene cluster was determined. This gene set contains Fibrillin, Fibroblast Activation Protein alpha, six Collagen protein subunits, and Versican, which are genes/proteins that are typically produced by fibroblast/mesenchymal cells (Ross et al., Nat. Genet. 24:227-35, 2000). This analysis showed that the fibroblast/mesenchymal profile was correlated with intrinsic subtype (Table 3, p=0.012) and that the MetScore 3 samples had the lowest expression compared to the MetScore 1 and 2 samples. Pathological examination of hematoxylin and eosin (H&E) sections of the distant metastasis samples also supported this conclusion and revealed scant admixed mesenchymal cells in the distant metastasis samples versus their primaries, which show abundant admixed mesenchymal cells.

### Correlations Between Tumor Subtype, VEGF-Profile And Other Metastasis Associated Profiles

Many different expression-based predictors for breast cancer patient outcomes have been developed, and in some cases, the time to metastasis development has been used as the supervising endpoint. Therefore, using the training data set, an examination was made to determine whether the previously defined tumor intrinsic subtypes, the MetScore classification and the VEGF-signature correlated with any of the following expression profiles: A) the NKI 70-gene outcome predictor (van de Vijver et al., N. Engl. J. Med. 347:1999-2009, 2002; van't Veer et al., Nature 415:530-36, 2002); B) the “wound-response” profile (Chang et al., PLoS Biol. 2:E7, 2004); C) the cell line derived hypoxia-induced profile (Chi et al., PLoS Med. 3:e47, 2006); D) the 11-gene BMI/stem cell signature (Glinsky et al., J. Clin. Invest. 115:1503-21, 2005); E) a bone metastasis signature (Kang et al., Cancer Cell 3:537-49, 2003); F) a lung metastasis signature (Minn et al., Nature 436:518-24, 2005); and G) the expression profile of three individual genes (HIF1α, Snail (Moody et al., Cancer Cell 8:197-209, 2005) and Twist (Yang et al., Cell 117:927-39, 2004)).

These analyses identified a large amount of concordance between profiles (Table 3), showing that they are tracking a common set of biological phenotypes. For example, breast tumor subtype was significantly correlated with the Bone and Lung Metastasis profiles, Snail expression, and the 11-gene stem cell signature. In particular, the bone and lung profiles were associated with both ER-negative subtypes (Basal-like and HER2+/ER−), and Snail expression and the 11-gene stem cell signature were the highest within the Basal-like subtype. Similar results were also observed when the VEGF-profile was compared to the other profiles, and in all cases, the high expression of the VEGF-profile correlated with the high expression of the other signatures whose high expression predicts a poor outcome. A “hypoxia signature” was recently identified using cell lines, and shown to be of prognostic value across a variety of tumor types including breast (Chi et al., PLoS Med. 3:e47, 2006). This large signature showed a four gene overlap with the 13-gene VEGF-profile (ADM, NDRG1, DDIT4, and ANGPLT4). The correlation between the cell line “hypoxia signature” and the 13-gene VEGF-profile was statistically significant (Table 3; p<0.001). However, the lack of VEGF and SLC16A3 in the cell line signature showed that these are related, but distinct signatures.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for evaluating the prognosis of a breast cancer patient, comprising determining the expression levels of the RNA transcripts or their expression products of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample comprising a cancer cell from said patient, normalized against the expression levels of all RNA transcripts or their expression products in said sample, or of a reference set of RNA transcripts or their expression products in said sample, wherein expression of said biomarkers is indicative of prognosis, thereby evaluating the prognosis of said breast cancer patient.

2. The method of claim 1, wherein overexpression of said biomarkers is indicative of a poor prognosis.

3. The method of claim 1, wherein absence of overexpression of said biomarkers is indicative of a good prognosis.

4. The method of claim 1, wherein detecting expression of said biomarkers comprises performing nucleic acid hybridization, quantitative RT-PCR or immunohistochemistry.

5. The method of claim 1, wherein said method for evaluating the prognosis of a breast cancer patient further comprises assessment of clinical information.

6. The method of claim 5, wherein said clinical information comprises tumor size, tumor grade, lymph node status, and family history.

7. The method of claim 6, wherein said method is used to develop a treatment strategy for said breast cancer patient.

8. The method of claim 1, wherein said method for evaluating the prognosis of a breast cancer patient is coupled with analysis of Her-2 expression levels.

9. The method of claim 1, wherein said method for evaluating the prognosis of a breast cancer patient is coupled with analysis of estrogen receptor or progesterone receptor status of said patient.

10. The method of claim 1, wherein said method for evaluating the prognosis of a breast cancer patient is independent of estrogen receptor status of said patient.

11. The method of claim 1, wherein said method is used to evaluate the prognosis of an estrogen receptor-positive or an estrogen receptor-negative breast cancer patient.

12. The method of claim 1, wherein said RNA is isolated from a fixed, paraffin-embedded sample comprising a cancer cell from said patient.

13. The method of claim 1, wherein said RNA is isolated from core biopsy tissue or fine needle aspirate cells comprising a cancer cell from said patient.

14. A method for evaluating the prognosis of a breast cancer patient, comprising determining the expression levels of the RNA transcripts of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample comprising a cancer cell from said patient, normalized against the expression levels of all RNA transcripts in said sample, wherein overexpression of said biomarkers is indicative of a poor prognosis, thereby evaluating the prognosis of said breast cancer patient.

15. A method for evaluating the prognosis of a breast cancer patient, comprising determining the expression levels of the RNA transcripts of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample comprising a cancer cell from said patient, normalized against the expression levels of a reference set of RNA transcripts in said sample, wherein overexpression of said biomarkers is indicative of a poor prognosis, thereby evaluating the prognosis of said breast cancer patient.

16. A method for predicting a response of a breast cancer patient to a selected treatment, comprising determining the expression levels of the RNA transcripts or their expression products of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample comprising a cancer cell from said patient, normalized against the expression levels of all RNA transcripts or their expression products in said sample, or of a reference set of RNA transcripts or their expression products in said sample, wherein overexpression of said biomarkers is indicative of a positive treatment response, thereby predicting the response of said breast cancer patient to said treatment.

17. The method of claim 16, wherein said treatment comprises anti-VEGF therapy.

18. The method of claim 17, wherein said anti-VEGF therapy comprises a monoclonal antibody.

19. The method of claim 18, wherein said monoclonal antibody is bevacizumab.

20. The method of claim 16, wherein said treatment comprises anti-HIFla therapy.

21. A method for evaluating the prognosis of a breast cancer patient, comprising detecting expression of at least five biomarkers selected from the group consisting of RRAGD, FABP5, UCHL1, GAL, PLOD, DDIT4, VEGF, ADM, ANGPTL4, NDRG1, NP, SLC16A3, and C14ORF58 in a sample from said patient, wherein overexpression of said biomarkers is indicative of a poor prognosis, thereby evaluating the prognosis of said breast cancer patient.

22. A method for evaluating the prognosis of a breast cancer patient, comprising determining the expression levels of the RNA transcripts or their expression products of a set of biomarkers comprising RRAGD, FABP5, UCHL1, GAL, PLOD, DDIT4, VEGF, ADM, ANGPTL4, NDRG1, NP, SLC16A3, and C14ORF58 in a sample comprising a cancer cell from said patient, normalized against the expression levels of all RNA transcripts or their expression products in said sample, or of a reference set of RNA transcripts or their expression products in said sample, wherein expression of said set of biomarkers is indicative of prognosis, thereby evaluating the prognosis of said breast cancer patient.

23. The method of claim 22, wherein overexpression of said set of biomarkers is indicative of a poor prognosis.

24. The method of claim 22, wherein absence of overexpression of said set of biomarkers is indicative of a good prognosis.

25. A kit comprising at least five nucleic acid probes, wherein each of said probes specifically binds to one of five distinct biomarker nucleic acids or fragments thereof selected from the group consisting of RRAGD, FABP5, UCHL1, GAL, PLOD, DDIT4, VEGF, ADM, ANGPTL4, NDRG1, NP, SLC16A3, and C14ORF58.
